(19) United States
(12) Patent Application Publication
(10) Pub. No.: US 2022/0301839 A1
(43) Pub. Date: Sep. 22, 2022

(54) METHOD FOR ANALYZING MASS SPECTROMETRY DATA, COMPUTER PROGRAM MEDIUM, AND DEVICE FOR ANALYZING MASS SPECTROMETRY DATA

(71) Applicant: TOYOTA JIDOSHA KABUSHIKI KAISHA, Toyota-shi (JP)

(72) Inventors: Takahiro Tsubouchi, Miyoshi-shi (JP); Hideo Kato, Nagoya-shi (JP)

(21) Appl. No.: 17/655,129

(22) Filed: Mar. 16, 2022

(30) Foreign Application Priority Data
Mar. 16, 2021 (JP) 2021-042862

(51) Int. Cl. H01J 49/00; G16C 20/20

(52) U.S. Cl. CPC H01J 49/0036; G16C 20/20

(57) ABSTRACT

One aspect of the present disclosure relates to a method for analyzing mass spectrometry data of an analysis target substance comprising: acquiring mass-to-charge ratios and relative intensities of one or more detected ion peaks; preparing mass data of the analysis target substance consisting of the relative intensities, the mass-to-charge ratios, and differences in the mass-to-charge ratios of the one or more ion peaks; and determining an identification candidate for the analysis target substance or partial structure thereof. Another aspect of the present disclosure relates to a computer program medium storing a computer program for executing the method for analyzing mass spectrometry data of an analysis target substance, and a device for analyzing mass spectrometry data of an analysis target substance.

### CROSS REFERENCE TO RELATED APPLICATIONS

The present application claims priority from Japanese patent application JP 2021-042862 filed on Mar. 16, 2021, the entire content of which is hereby incorporated by reference into this application.

### BACKGROUND

### Technical Field

The present disclosure relates to a method for analyzing mass spectrometry data, a computer program medium storing a program for executing the method for analyzing mass spectrometry data of an analysis target substance, and a device for analyzing mass spectrometry data of an analysis target substance.

### Background Art

Mass spectrometry has been widely used as means to identify an analysis target substance in various kinds of technical fields.

For example, JP 2007-285719 A discloses a method for analyzing mass spectrometry data in which precursor ions derived from each component of one or a plurality of unknown components are cleaved in n−1 (n≥2) phases, and a component similar to an objective component is selected among the unknown components based on results of an MSⁿ analysis that performs mass spectrometry on the generated fragment ions. In the method, a predetermined variable is derived from spectral data acquired by the MSⁿ analysis and MSⁿ spectral data of the objective component to obtain a degree of similarity of the objective component with each unknown component by multivariate analysis using the variable and the component similar to the objective component is selected based on the degree of similarity.

WO 2008/065704 discloses a device for analyzing mass spectrometry data that separates respective samples before and after a chemical change by chromatograph and after that performs a MS measurement and a MSMS measurement by a mass spectrometer to search a product brought by the chemical change based on the acquired data. The mass spectrometry data analysis device includes a product search unit that compares the samples before and after the chemical change in chromatograms of mass-to-charge ratios of precursor ions in the MSMS measurement and determines a peak present only in the sample after the change as a peak derived from the product.

WO 2018/087824 discloses a device for analyzing data for chromatograph mass spectrometry that analyses data collected by a chromatograph mass spectrometer that includes a chromatograph that temporally separates components in a sample, a main detector as a mass spectrometer that detects components in the sample including the components separated by the chromatograph, and a sub-detector different from the mass spectrometer that detects components in the sample including the components separated by the chromatograph. The device includes an arithmetic processing unit and a mass-to-charge ratio extraction unit.

### SUMMARY

Conventionally, mass spectrometry data to identify an analysis target substance has been analyzed by comparing a mass spectrum of the analysis target substance with a mass spectrometry database of the already-known compounds prepared based on mass spectra of the already-known compounds and determining an identification candidate from a degree of match of spectrum patterns. Since the conventional analysis method needs to use a high-resolution mass spectrometer that can perform isotope identification, there has been a problem of high economical cost required for the analysis. Additionally, since the conventional analysis method determines an identification candidate from a degree of match of spectrum patterns, in a case where data of an analysis target substance contained in a sample is not registered with a mass spectrometry database of already-known compounds, there has been also a problem that, in addition to determination of an identification candidate being difficult, acquiring information on the partial structure is difficult. When a low resolution mass spectrometer, which allows low-cost analysis, is used, and/or data of an analysis target substance contained in a sample is not registered with a mass spectrometry database of already-known compounds, for determination of an identification candidate and/or acquisition of information on the partial structure, analysis of the mass spectrometry data needs to depend on an experience value of an analyst having rich experience. This raised a problem of high personal and temporal costs required for analysis.

Accordingly, the present disclosure provides a means for analyzing mass spectrometry data of an analysis target substance that allows determining an identification candidate and/or acquiring information on a partial structure at low cost.

The inventors have variously examined means for solving the problems. The inventors have found it possible to determine an identification candidate for an analysis target substance or partial structure thereof by acquiring mass-to-charge ratios and relative intensities of one or more detected ion peaks by measuring a mass spectrum of the analysis target substance; preparing mass data of the analysis target substance consisting of the relative intensities, the mass-to-charge ratios, and differences in the mass-to-charge ratios of the one or more ion peaks by calculating the difference in the mass-to-charge ratios between the ion peak and another ion peak for each of the one or more detected ion peaks; and comparing the mass data with mass data of one or more reference substances. The inventors have completed the present disclosure based on the finding.

That is, the present disclosure encompasses the following aspects and embodiments.

(1) A method for analyzing mass spectrometry data of an analysis target substance comprising:

acquiring mass-to-charge ratios and relative intensities of one or more detected ion peaks by measuring a mass spectrum of the analysis target substance;

preparing mass data of the analysis target substance consisting of the relative intensities, the mass-to-charge ratios, and differences in the mass-to-charge ratios of the one or more ion peaks by calculating the difference in the mass-to-charge ratios between the ion peak and another ion peak for each of the one or more detected ion peaks; and

determining an identification candidate for the analysis target substance or partial structure thereof by comparing the prepared mass data of the analysis target substance with mass data of one or more reference substances or partial structure thereof.

(2) The method according to the embodiment (1), further comprising:

preparing mass data of the reference substance or partial structure thereof consisting of relative intensities, mass-to-charge ratios, and differences in the mass-to-charge ratios of one or more ion peaks for each of the one or more reference substances by measuring a mass spectrum of the one or more reference substances and calculating the difference in the mass-to-charge ratios between the one or more detected ion peaks and another ion peak for each of the one or more reference substances.

(3) The method according to the embodiment (1) or (2),

wherein the determining further comprises:

determining the identification candidates for the analysis target substance or partial structure thereof by setting a threshold n (note that n≥0) of the relative intensity of the ion peak, and comparing mass data of the analysis target substance consisting of the relative intensities of the threshold n or more, the mass-to-charge ratios, and the differences in the mass-to-charge ratios with the mass data of the one or more reference substances or partial structure thereof; and

determining a ranking of the identification candidates as having increased reliability by performing the determining of the identification candidates for the analysis target substance or partial structure thereof repeatedly while changing the threshold n within a range of 0 or more to a maximum value of the relative intensities of the one or more detected ion peaks, and selecting the identification candidate determined based on larger number of ion peaks and the identification candidate determined in case that larger value of the threshold n is set.

(4) A computer program medium storing a computer program for executing the method for analyzing mass spectrometry data of an analysis target substance according to any one of the embodiments (1) to (3).

(5) A device for analyzing mass spectrometry data of an analysis target substance comprising:

a mass spectrum measuring unit that measures a mass spectrum of the analysis target substance;

an analysis target substance data element acquiring unit that acquires mass-to-charge ratios and relative intensities of one or more detected ion peaks in the mass spectrum measuring unit;

an analysis target substance mass data preparing unit that prepares mass data of the analysis target substance consisting of the relative intensities, the mass-to-charge ratios, and differences in the mass-to-charge ratios of the one or more ion peaks by calculating the difference in the mass-to-charge ratios between the ion peak and another ion peak for each of the one or more detected ion peaks; and

an analysis target substance identification candidate determining unit that determines an identification candidate for the analysis target substance or partial structure thereof by comparing the prepared mass data of the analysis target substance with mass data of one or more reference substances or partial structure thereof.

The present disclosure can provide analysis means of the mass spectrometry data of an analysis target substance that allows determining an identification candidate and/or acquire information on the partial structure at low cost.

### BRIEF DESCRIPTION OF THE DRAWINGS

FIG. 1 is a flowchart depicting respective steps according to one embodiment of a method for analyzing mass spectrometry data of an analysis target substance according to one aspect of the present disclosure;

FIG. 2 is a flowchart depicting respective steps according to one specific embodiment of a method for analyzing mass spectrometry data of an analysis target substance according to one aspect of the present disclosure; and

FIG. 3 is a mass spectrum of an analysis target substance measured in Experiment I-2.

### DETAILED DESCRIPTION

The following will describe embodiments of the present disclosure in detail.

<1. Method for Analyzing Mass Spectrometry Data of Analysis Target Substance>

The inventors have found it possible to determine an identification candidate for an analysis target substance or partial structure thereof by acquiring mass-to-charge ratios and relative intensities of one or more detected ion peaks by measuring a mass spectrum of the analysis target substance; preparing mass data of the analysis target substance consisting of the relative intensities, the mass-to-charge ratios, and differences in the mass-to-charge ratios of the one or more ion peaks by calculating the difference in the mass-to-charge ratios between the ion peak and another ion peak for each of the one or more detected ion peaks; and comparing the mass data with mass data of one or more reference substances. Accordingly, one aspect of the present disclosure relates to a method for analyzing mass spectrometry data of an analysis target substance.

The respective aspects of the present disclosure are applicable to, not only a high-resolution mass spectrometer that can perform isotope identification, but also a low-resolution mass spectrometer, which allows low-cost analysis. Usually, a mass spectrometer to which the respective aspects of the present disclosure are applied only needs to have a mass resolution of 100 or more. Embodying the respective aspects of the present disclosure using the low-resolution mass spectrometer allows determining an identification candidate for the analysis target substance and/or acquiring information on a partial structure thereof at low cost.

The respective aspects of the present disclosure are applicable to various kinds of mass spectrometers that include ion sources and mass analyzers usually used in the technical field. The mass spectrometer to which the respective aspects of the present disclosure are applied may have a configuration of a chromatograph mass spectrometer, such as a liquid chromatograph mass spectrometer or a gas chromatograph mass spectrometer.

In the respective aspects of the present disclosure, an analysis target substance may have a form of a single substance that has been preliminarily purified and isolated, or may have a form of a mixture that contains one or more other substances together. As will be described later, the respective aspects of the present disclosure feature in that mass data of an analysis target substance consisting of relative intensities, mass-to-charge ratios, and differences in the mass-to-charge ratios of ion peaks of the analysis target substance is compared with mass data of one or more reference substances. In view of this, even when the analysis target substance is in a form of a mixture, an identification candidate for the analysis target substance or partial structure thereof can be easily determined. In the case of the analysis target substance being in a form of a mixture, the mass spectrometer to which the respective aspects of the present disclosure are applied has a configuration of a chromatograph mass spectrometer in some embodiments.

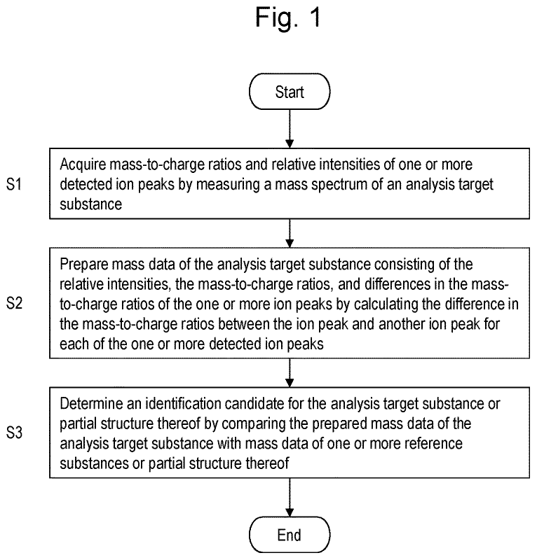

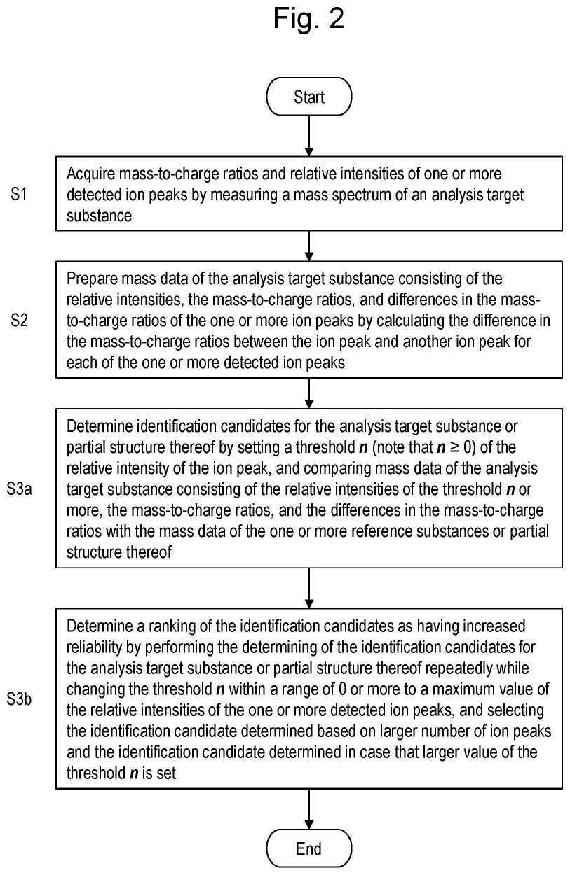

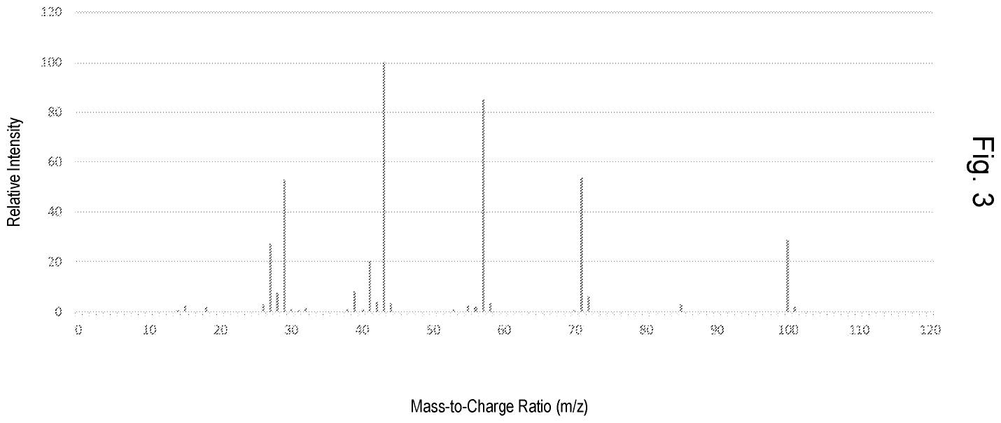

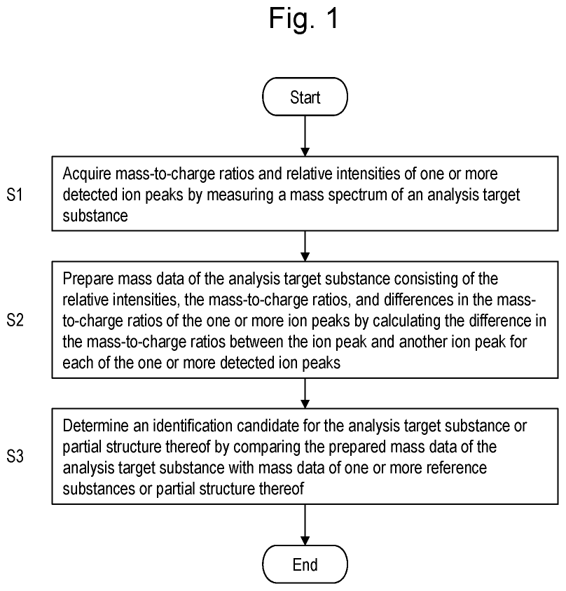

FIG. 1 depicts a flowchart showing respective steps in one embodiment of the method of this aspect. As depicted in FIG. 1, the method of this aspect includes an analysis target substance data element acquiring step (Step S1), an analysis target substance mass data preparing step (Step S2), and an analysis target substance identification candidate determining step (Step S3). The following will describe each step in detail.

[1-1. Analysis Target Substance Data Element Acquiring Step]

This step (Step S1) includes acquiring mass-to-charge ratios and relative intensities of one or more detected ion peaks by measuring a mass spectrum of an analysis target substance.

In this step, the mass spectrum of the analysis target substance can be measured using the various kinds of mass spectrometers described above as an example. Usually, the number of ion peaks of the analysis target substance for acquiring data elements consisting of mass-to-charge ratios and relative intensities possibly varies based on, not only a structure of the analysis target substance, but also measuring conditions of the mass spectrum or the like. In view of this, the mass spectrum of the analysis target substance is measured under a condition in which the number of detected ion peaks increase in some embodiments. Acquiring mass-to-charge ratios and relative intensities of increased number of ion peaks allows improvement in reliability in the determination of an identification candidate for the analysis target substance or partial structure thereof.

[1-2. Analysis Target Substance Mass Data Preparing Step]

This step (Step S2) includes preparing mass data of the analysis target substance consisting of the relative intensities, the mass-to-charge ratios, and differences in the mass-to-charge ratios of the one or more ion peaks by calculating the difference in the mass-to-charge ratios between the ion peak and another ion peak for each of the one or more ion peaks detected in the analysis target substance data element acquiring step (Step S1).

In this step, the mass data of the analysis target substance can be prepared by, for example, the following procedure. The mass-to-charge ratios and the relative intensities of the one or more ion peaks acquired in the analysis target substance data element acquiring step are output from the mass spectrometer and are input to respective cells at the first row and the first column in a spreadsheet formed of a plurality of rows and columns. In the spreadsheet, the difference in the mass-to-charge ratios between each ion peak and another ion peak is output to a cell corresponding to the row of the ion peak and the column of the other ion peak. In the method of this aspect, a difference of 0 or less does not contribute to determination of an identification candidate for the analysis target substance or partial structure thereof. In view of this, in this embodiment, in the case of the difference being 0 or less, 0 or a null is output to the corresponding cell in some embodiments.

[1-3. Analysis Target Substance Identification Candidate Determining Step]

This step (Step S3) includes determining an identification candidate for the analysis target substance or partial structure thereof by comparing the mass data of the analysis target substance prepared in the analysis target substance mass data preparing step (Step S2) with mass data of one or more reference substances or partial structure thereof.

There may be a case where, in mass spectrometry of a substance, ions (parent ions) generated by ionization of the substance are cleaved to generate charged fragment ions and neutral fragments (fragmentation). Patterns of the parent ions and the fragment ions generated by the fragmentation are closely related to a structure of the substance. In view of this, in this step, the mass data of the analysis target substance consisting of the relative intensities, the mass-to-charge ratios, and the differences in the mass-to-charge ratios of the one or more ion peaks is compared with mass data of one or more reference substances or partial structure thereof. Thus, compared with the related art that determines an identification candidate from a degree of match of spectrum patterns, the present disclosure allows further easily determining an identification candidate for the analysis target substance or partial structure thereof.

In this step, the mass data compared to determine an identification candidate for the analysis target substance or partial structure thereof is a combination of values of the mass-to-charge ratios and values of the differences in the mass-to-charge ratios of the one or more ion peaks in some embodiments. In a fragmentation of mass spectrometry, a difference in mass-to-charge ratios of a parent ion peak and a fragment ion peak corresponds to a mass of the neutral fragment. Here, which of the fragments charges varies based on a structure of the parent ion, conditions for ionization, or the like in some cases. In view of this, the mass-to-charge ratios and the differences in the mass-to-charge ratios of the mass data of the analysis target substance are compared with mass-to-charge ratios and differences in the mass-to-charge ratios of mass data of one or more reference substances or partial structure thereof, and in a case where a combination of a value of the mass-to-charge ratio and a value of the difference in the mass-to-charge ratios of the mass data of the analysis target substance matches a combination of a value of the mass-to-charge ratio and a value of the difference in the mass-to-charge ratios of mass data of a specific reference substance or partial structure thereof, the reference substance or partial structure thereof is determined as an identification candidate for the analysis target substance or partial structure thereof in some embodiments. For example, in a case where a value of the mass-to-charge ratio of the mass data of the analysis target substance is a and a value of the difference in the mass-to-charge ratios is b, when a value of the mass-to-charge ratio of mass data of a specific reference substance or partial structure thereof is a or b and a value of the difference in the mass-to-charge ratios is b or a, it can be determined that the combination of the value of the mass-to-charge ratio and the value of the difference in the mass-to-charge ratios of the mass data of the analysis target substance matches the combination of the value of the mass-to-charge ratio and the value of the difference in the mass-to-charge ratios of the mass data of the specific reference substance or its partial structure. Performing this step in the procedure allows determination of an identification candidate for the analysis target substance or partial structure thereof more easily compared with the related art that determines an identification candidate from a degree of match of spectrum patterns.

In this step, the mass data of the one or more reference substances or partial structure thereof may be prepared each time that this step is performed, or the preliminarily prepared mass data may be used. The mass data of the one or more reference substances or partial structure thereof used in this step can be prepared by performing a reference substance or partial structure thereof mass data preparing step that will be described below. In this step, the preliminarily prepared mass data of the one or more reference substances or partial structure thereof is used in some embodiments. The preliminarily prepared mass data of the one or more reference substances or partial structure thereof is stored in a reference substance data storage unit (for example, a storage device in a computer) in a data analysis device, and is output from the reference substance data storage unit for use when this step is performed in some embodiments. In the case of this embodiment, the period during which the method of this aspect is performed can be shortened and the cost can be lowered.

This step can be performed by, for example, the following procedure. Values of the mass-to-charge ratios and values of the differences in the mass-to-charge ratios output to the column of each ion peak in the spreadsheet prepared in the analysis target substance mass data preparing step are compared with values of the mass-to-charge ratios and values of the differences in the mass-to-charge ratios of the mass data of the one or more reference substances or partial structure thereof. When combinations of the values of the mass-to-charge ratios and the values of the differences in the mass-to-charge ratios of both are matched, a name of the reference substance matched is output to the cell for an identification candidate in the column. In the case of this embodiment, as the mass data of the one or more reference substances or partial structure thereof, the mass data preliminarily stored in a reference substance data storage unit (for example, a storage device in a computer) in a data analysis device may be used by outputting it to a spreadsheet different from the spreadsheet to which the mass data of the analysis target substance has been output.

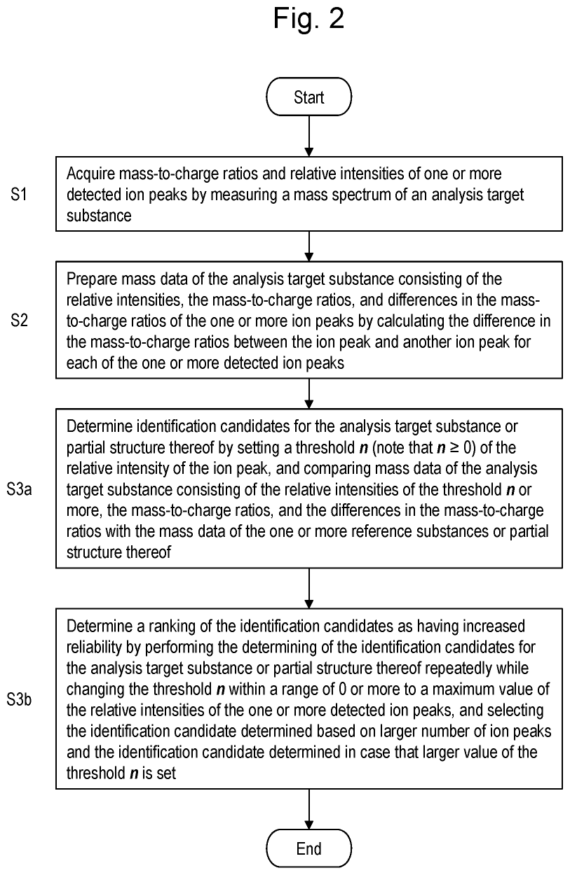

This step further includes a step of determining the identification candidates for the analysis target substance or partial structure thereof by setting a threshold n (note that n≥0) of the relative intensity of the ion peak, and comparing the mass data of the analysis target substance consisting of the relative intensities of the threshold n or more, the mass-to-charge ratios, and the differences in the mass-to-charge ratios with the mass data of the one or more reference substances or partial structure thereof (Step S3a), and determining a ranking of the identification candidates as having increased reliability by performing the steps repeatedly while changing the threshold n (Step S3b) in some embodiments. FIG. 2 depicts the flowchart showing the respective steps in this embodiment.

In a mass spectrum, a relative intensity of an ion peak usually associates with an abundance ratio of the ion. In view of this, fragment ions generated by fragmentation usually have relative intensities to the same extent of the parent ion. Additionally, ion peaks having increased relative intensity are usually caused by increased number of ions. Accordingly, in this embodiment, the threshold n of the relative intensity of the ion peak is set to narrow down the mass data to the mass data of the analysis target substance consisting of the relative intensities of the threshold n or more, the mass-to-charge ratios, and the differences in the mass-to-charge ratios, and the threshold n is changed for repeated comparison with the mass data of the one or more reference substances or partial structure thereof, thus allowing determining an identification candidate for the analysis target substance or partial structure thereof with further high reliability.

In this embodiment, the threshold n of the relative intensity of the ion peak is changed within a range of 0 or more to the maximum value of the relative intensities of the one or more detected ion peaks. In this case, the change width of threshold n can be appropriately set based on, for example, the number of ion peaks and the maximum value of the relative intensities. The change width of the threshold n is usually a value of 1 or more and is an integer of 1 or more in some embodiments. The number of repetitions due to the change in the threshold n is usually two times or more and within a range of from two to 10 times in some embodiments. The change width of the threshold n being less than the lower limit value causes a possibility that the result actually the same as the result before the change is acquired. The number of repetitions due to the change in the threshold n being less than the lower limit value causes a possibility that the result actually the same as the result when the repetition is not performed is acquired. Moreover, the number of repetitions due to the change in the threshold n in excess of the upper limit value causes a possibility that the period during which the method of this aspect is performed increases. Accordingly, performing the method of this embodiment with the condition allows determining an identification candidate for the analysis target substance or partial structure thereof at low cost and with further higher reliability.

As described above, in a mass spectrum, a relative intensity of an ion peak usually associates with an abundance ratio of the ion. In view of this, a ranking of identification candidates can be determined as having increased reliability by selecting the identification candidate determined based on larger number of ion peaks, and the identification candidate determined in case that larger value of the threshold n is set. Determining a ranking of the identification candidates based on the number of ion peaks and the value of the threshold n allows improvement in the determination result of the identification candidates.

The embodiment can be performed by, for example, the following procedure. The threshold n of the relative intensity of the ion peak is set. In the spreadsheet prepared in the analysis target substance mass data preparing step, rows are narrowed down to only the rows in which the ion peaks have the relative intensities of the threshold n or more, and the rows are output. Values of the mass-to-charge ratios and values of the differences in the mass-to-charge ratios output to the columns of the narrowed ion peaks are compared with values of the mass-to-charge ratios and values of the differences in the mass-to-charge ratios of the mass data of the one or more reference substances or partial structure thereof. When combinations of the values of the mass-to-charge ratios and the values of the differences in the mass-to-charge ratios of both are matched, a name of the reference substance matched is output to a cell for an identification candidate in the column. The threshold n is changed within the range of from 0 or more to the maximum value of the relative intensity of the one or more detected ion peaks, and the step is repeatedly performed. After having ended the repetition, the names of the reference substances output to the cells for the identification candidates in the columns of larger number of ion peaks, and the names of the reference substances output to the cells for the identification candidates determined in case that larger value of the threshold n is set are ranked as the identification candidates as having the increased reliability. The names and the ranking of the reference substances are output to the spreadsheet.

[1-4. Reference Substance or Partial Structure Thereof Mass Data Preparing Step]

The method of this aspect may include a reference substance or partial structure thereof mass data preparing step as desired. For example, in the case where the mass data of the one or more reference substances or partial structure thereof is not prepared and/or the method of this aspect is performed using the preliminarily prepared mass data of the one or more reference substances or partial structure thereof but an identification candidate for the analysis target substance or partial structure thereof cannot be successfully determined, this step is included in some embodiments.

This step includes preparing mass data of the reference substance or partial structure thereof consisting of relative intensities, mass-to-charge ratios, and differences in the mass-to-charge ratios of one or more ion peaks for each of the one or more reference substances by measuring a mass spectrum of the one or more reference substances and calculating the difference in the mass-to-charge ratios between the one or more detected ion peaks and another ion peak for each of the one or more reference substances.

In the respective aspects of the present disclosure, the number of reference substances and the kind of the reference substance are not specifically limited. The number of reference substances and the kind of the reference substance can be appropriately selected based on a structure and/or physicochemical properties of the analysis target substance. Performing the method of this aspect using the reference substances having a structure and/or physicochemical properties similar to the structure and/or the physicochemical properties of the analysis target substance can improve the reliability in the determination of an identification candidate for the analysis target substance or partial structure thereof.

This step can be performed by the procedure similar to the analysis target substance mass data preparing step described above.

In this step, the mass data of the one or more reference substances or partial structure thereof can be prepared, for example, by the following procedure. Mass spectra of the one or more reference substances are each measured to acquire the mass-to-charge ratios and the relative intensities of the one or more detected ion peaks. The acquired mass-to-charge ratios and relative intensities of the one or more ion peaks are input to respective cells at the first row and the first column in the spreadsheet formed of a plurality of rows and columns, and the differences in the mass-to-charge ratios between each ion peak and another ion peak are output to a cell corresponding to the row of the ion peak and the column of the other ion peak. In the method of this aspect, a difference of 0 or less does not contribute to the determination of an identification candidate for the analysis target substance or partial structure thereof. In view of this, in this embodiment, in the case of the difference being 0 or less, 0 or a null is output to the corresponding cell in some embodiments.

The mass data of the one or more reference substances or partial structure thereof prepared in the procedure is stored in a reference substance data storage unit (for example, a storage device in a computer) in a data analysis device, and is output from the reference substance data storage unit for use when the analysis target substance identification candidate determining step is performed in some embodiments. In the case of this embodiment, the period during which the method of this aspect is performed can be shortened and the cost can be lowered.

<2. Computer Program for Executing Method for Analyzing Mass Spectrometry Data>

Another aspect of the present disclosure relates to a computer program for executing the method for analyzing mass spectrometry data. Another aspect of the present disclosure relates to a computer program medium storing the computer program for executing the method for analyzing mass spectrometry data.

The program of this aspect is usable to perform the method for analyzing mass spectrometry data of the analysis target substance according to one aspect of the present disclosure on a data analysis device (for example, a computer). The program of this aspect includes: the analysis target substance data element acquiring step, the analysis target substance mass data preparing step, and the analysis target substance identification candidate determining step. Each step corresponds to each step of the method for analyzing mass spectrometry data of the analysis target substance according to one aspect of the present disclosure described above.

<3. Device for Analyzing Mass Spectrometry Data of Analysis Target Substance>

Another aspect of the present disclosure relates to a device for analyzing mass spectrometry data of an analysis target substance. As described above, the method for analyzing mass spectrometry data of an analysis target substance according to one aspect of the present disclosure allows determining an identification candidate and/or acquiring information on partial structure thereof at low cost. Accordingly, analyzing mass spectrometry data of an analysis target substance using the device according to this aspect allows determining an identification candidate and/or acquiring information on partial structure thereof at low cost.

The device according to this aspect comprises:

a mass spectrum measuring unit that measures a mass spectrum of an analysis target substance;

an analysis target substance data element acquiring unit that acquires a mass-to-charge ratios and relative intensities of one or more detected ion peaks in the mass spectrum measuring unit;

an analysis target substance mass data preparing unit that prepares mass data of the analysis target substance consisting of the relative intensities, the mass-to-charge ratios, and differences in the mass-to-charge ratios of the one or more ion peaks by calculating the difference in the mass-to-charge ratios between the ion peak and another ion peak for each of the one or more detected ion peaks; and

an analysis target substance identification candidate determining unit that determines an identification candidate for the analysis target substance or partial structure thereof by comparing the prepared mass data of the analysis target substance with mass data of one or more reference substances or partial structure thereof.

In the device according to this aspect, the mass spectrum measuring unit may be the mass spectrometer described above as an example.

In the device according to this aspect, the analysis target substance data element acquiring unit, the analysis target substance mass data preparing unit, and the analysis target substance identification candidate determining unit may be units included in a data analysis device usually used for analysis of mass spectrometry data in the technical field. The data analysis device usually includes a central control unit, a spectral data generating unit, an analysis processing unit, a measurement data storage unit, a reference substance data storage unit, an input unit, and an output unit. The data analysis device has, for example, a configuration of a computer that includes a central processing unit, a storage device such as a hard drive, an input device, and an output device in some embodiments. In this case, the computer program according to one aspect of the present disclosure as well as a control program for a mass spectrometer are preliminarily installed on a computer, and the method for analyzing mass spectrometry data of an analysis target substance according to one aspect of the present disclosure is performed. Thus, the measurement of mass spectrum of the analysis target substance and the determination of an identification candidate and/or the acquisition of information of partial structure thereof can be easily achieved at low cost.

### EXAMPLES

The following will further specifically describe the present disclosure using examples. However, the technical scope of the present disclosure is not limited to the examples.

<I: Method for Analyzing Mass Spectrometry Data of Analysis Target Substance>

### [I-1: Mass Data Preparation of Reference Substance]

Mass spectra of various kinds of reference substances were measured to acquire mass-to-charge ratios (m/z) and intensities of one or more detected ion peaks for each of one or more reference substances. Differences in the mass-to-charge ratios between each ion peak and another ion peak were calculated for each of the one or more detected ion peaks to prepare mass data of the reference substances or partial structures thereof consisting of the relative intensities, the mass-to-charge ratios, and the differences in the mass-to-charge ratios of the one or more ion peaks for each of the one or more reference substances (the reference substance or partial structure thereof mass data preparing step). Table 1 shows the mass data of the reference substances.

TABLE 1

| Reference substances or | Fragment | Differences in mass-to-charge ratio | | | | |
|---|---|---|---|---|---|---|
| partial structures thereof | symbol | 1 | 2 | 3 | 4 | 5 |
| Alkane | A | 43 | 51 | | | |
| Alkene | B | 41 | 55 | | | |
| Benzene ring | C | 77 | | | | |
| Benzyl group | D | 65 | 91 | | | |
| Aliphatic alcohol | E | 31 | 45 | | | |
| Aliphatic ketone | F | 43 | 57 | | | |
| Aliphatic carboxylic acid | G | 45 | 60 | 74 | 88 | |
| Phenol | H | 82 | 100 | | | |
| Benzyl alcohol | I | 77 | 79 | 107 | | |
| Aromatic carboxylic acid | J | 105 | 122 | | | |
| m- or p-phthalic acid | K | 121 | 149 | 166 | | |
| o-phthalic acid | L | 104 | 148 | | | |
| Aliphatic primary amine | M | 28 | 31 | 44 | 58 | |
| Aliphatic secondary | N | 42 | 43 | 44 | | |
| amine | | | | | | |
| Aliphatic amide | O | 29 | 44 | 58 | 72 | |
| Acrylic acid | P | 55 | 73 | | | |
| Methacrylic acid | Q | 69 | 87 | | | |
| Methyl ester | R | 59 | 74 | 88 | 102 | 143 |
| Acetic acid ester | S | 43 | 61 | 73 | | |

[I-2: Data Element Acquisition of Analysis Target Substance and Mass Data Preparation]

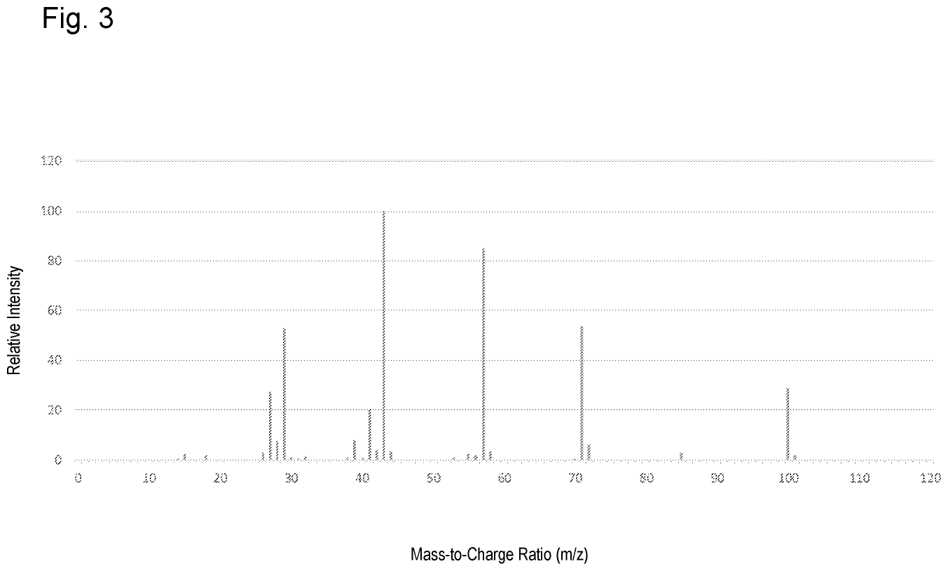

Mass spectrum of an analysis target substance was measured to acquire mass-to-charge ratios (m/z) and relative intensities of one or more detected ion peaks (the analysis target substance data element acquiring step). FIG. 3 illustrates the mass spectrum of the analysis target substance.

As illustrated in FIG. 3, in the mass spectrum of the analysis target substance, 28 ion peaks with mass-to-charge ratios (m/z) of 14 to 101 were detected. Differences in the mass-to-charge ratios between each ion peak and another ion peak were calculated for each of the detected 28 ion peaks to prepare mass data of the analysis target substance consisting of the relative intensities, the mass-to-charge ratios, and the differences in the mass-to-charge ratios of the 28 ion peaks (the analysis target substance mass data preparing step). Table 2 shows the prepared mass data. When the difference was 0 or less, 0 was output to the corresponding cell.

TABLE 2

| | | Relative intensities | | | | | | | | | |
|---|---|---|---|---|---|---|---|---|---|---|---|
| Relative | Mass-to-charge | 0.5 | 2.4 | 1.8 | 2.9 | 27.6 | 7.5 | 53 | 1.1 | 0.6 | 1.6 |
| intensities | ratios | 14 | 15 | 18 | 26 | 27 | 28 | 29 | 30 | 31 | 32 |
| 0.5 | 14 | 0 | 0 | 0 | 0 | 0 | 0 | 0 | 0 | 0 | 0 |
| 2.4 | 15 | 1 | 0 | 0 | 0 | 0 | 0 | 0 | 0 | 0 | 0 |
| 1.8 | 18 | 4 | 3 | 0 | 0 | 0 | 0 | 0 | 0 | 0 | 0 |
| 2.9 | 26 | 12 | 11 | 8 | 0 | 0 | 0 | 0 | 0 | 0 | 0 |
| 27.6 | 27 | 13 | 12 | 9 | 1 | 0 | 0 | 0 | 0 | 0 | 0 |
| 7.5 | 28 | 14 | 13 | 10 | 2 | 1 | 0 | 0 | 0 | 0 | 0 |
| 53 | 29 | 15 | 14 | 11 | 3 | 2 | 1 | 0 | 0 | 0 | 0 |
| 1.1 | 30 | 16 | 15 | 12 | 4 | 3 | 2 | 1 | 0 | 0 | 0 |
| 0.6 | 31 | 17 | 16 | 13 | 5 | 4 | 3 | 2 | 1 | 0 | 0 |
| 1.6 | 32 | 18 | 17 | 14 | 6 | 5 | 4 | 3 | 2 | 1 | 0 |
| 1 | 38 | 24 | 23 | 20 | 12 | 11 | 10 | 9 | 8 | 7 | 6 |
| 7.9 | 39 | 25 | 24 | 21 | 13 | 12 | 11 | 10 | 9 | 8 | 7 |
| 1.2 | 40 | 26 | 25 | 22 | 14 | 13 | 12 | 11 | 10 | 9 | 8 |
| 20.3 | 41 | 27 | 26 | 23 | 15 | 14 | 13 | 12 | 11 | 10 | 9 |
| 3.6 | 42 | 28 | 27 | 24 | 16 | 15 | 14 | 13 | 12 | 11 | 10 |
| 100 | 43 | 29 | 28 | 25 | 17 | 16 | 15 | 14 | 13 | 12 | 11 |
| 3.4 | 44 | 30 | 29 | 26 | 18 | 17 | 16 | 15 | 14 | 13 | 12 |
| 1 | 53 | 39 | 38 | 35 | 27 | 26 | 25 | 24 | 23 | 22 | 21 |
| 2.4 | 55 | 41 | 40 | 37 | 29 | 28 | 27 | 26 | 25 | 24 | 23 |
| 1.8 | 56 | 42 | 41 | 38 | 30 | 29 | 28 | 27 | 26 | 25 | 24 |
| 84.9 | 57 | 43 | 42 | 39 | 31 | 30 | 29 | 28 | 27 | 26 | 25 |
| 3.1 | 58 | 44 | 43 | 40 | 32 | 31 | 30 | 29 | 28 | 27 | 26 |
| 0.7 | 70 | 56 | 55 | 52 | 44 | 43 | 42 | 41 | 40 | 39 | 38 |
| 54 | 71 | 57 | 56 | 53 | 45 | 44 | 43 | 42 | 41 | 40 | 39 |
| 6.1 | 72 | 58 | 57 | 54 | 46 | 45 | 44 | 43 | 42 | 41 | 40 |
| 2.9 | 85 | 71 | 70 | 67 | 59 | 58 | 57 | 56 | 55 | 54 | 53 |
| 28.6 | 100 | 86 | 85 | 82 | 74 | 73 | 72 | 71 | 70 | 69 | 68 |
| 2 | 101 | 87 | 86 | 83 | 75 | 74 | 73 | 72 | 71 | 70 | 69 |

| | | Relative intensities | | | | | | | | |
|---|---|---|---|---|---|---|---|---|---|---|
| Relative | Mass-to-charge | 1 | 7.9 | 1.2 | 20.3 | 3.6 | 100 | 3.4 | 1 | 2.4 |
| intensities | ratios | 38 | 39 | 40 | 41 | 42 | 43 | 44 | 53 | 55 |
| 0.5 | 14 | 0 | 0 | 0 | 0 | 0 | 0 | 0 | 0 | 0 |
| 2.4 | 15 | 0 | 0 | 0 | 0 | 0 | 0 | 0 | 0 | 0 |
| 1.8 | 18 | 0 | 0 | 0 | 0 | 0 | 0 | 0 | 0 | 0 |
| 2.9 | 26 | 0 | 0 | 0 | 0 | 0 | 0 | 0 | 0 | 0 |
| 27.6 | 27 | 0 | 0 | 0 | 0 | 0 | 0 | 0 | 0 | 0 |
| 7.5 | 28 | 0 | 0 | 0 | 0 | 0 | 0 | 0 | 0 | 0 |
| 53 | 29 | 0 | 0 | 0 | 0 | 0 | 0 | 0 | 0 | 0 |
| 1.1 | 30 | 0 | 0 | 0 | 0 | 0 | 0 | 0 | 0 | 0 |
| 0.6 | 31 | 0 | 0 | 0 | 0 | 0 | 0 | 0 | 0 | 0 |
| 1.6 | 32 | 0 | 0 | 0 | 0 | 0 | 0 | 0 | 0 | 0 |
| 1 | 38 | 0 | 0 | 0 | 0 | 0 | 0 | 0 | 0 | 0 |
| 7.9 | 39 | 1 | 0 | 0 | 0 | 0 | 0 | 0 | 0 | 0 |
| 1.2 | 40 | 2 | 1 | 0 | 0 | 0 | 0 | 0 | 0 | 0 |
| 20.3 | 41 | 3 | 2 | 1 | 0 | 0 | 0 | 0 | 0 | 0 |
| 3.6 | 42 | 4 | 3 | 2 | 1 | 0 | 0 | 0 | 0 | 0 |
| 100 | 43 | 5 | 4 | 3 | 2 | 1 | 0 | 0 | 0 | 0 |
| 3.4 | 44 | 6 | 5 | 4 | 3 | 2 | 1 | 0 | 0 | 0 |
| 1 | 53 | 15 | 14 | 13 | 12 | 11 | 10 | 9 | 0 | 0 |
| 2.4 | 55 | 17 | 16 | 15 | 14 | 13 | 12 | 11 | 2 | 0 |
| 1.8 | 56 | 18 | 17 | 16 | 15 | 14 | 13 | 12 | 3 | 1 |
| 84.9 | 57 | 19 | 18 | 17 | 16 | 15 | 14 | 13 | 4 | 2 |
| 3.1 | 58 | 20 | 19 | 18 | 17 | 16 | 15 | 14 | 5 | 3 |
| 0.7 | 70 | 32 | 31 | 30 | 29 | 28 | 27 | 26 | 17 | 15 |
| 54 | 71 | 33 | 32 | 31 | 30 | 29 | 28 | 27 | 18 | 16 |
| 6.1 | 72 | 34 | 33 | 32 | 31 | 30 | 29 | 28 | 19 | 17 |
| 2.9 | 85 | 47 | 46 | 45 | 44 | 43 | 42 | 41 | 32 | 30 |
| 28.6 | 100 | 62 | 61 | 60 | 59 | 58 | 57 | 56 | 47 | 45 |
| 2 | 101 | 63 | 62 | 61 | 60 | 59 | 58 | 57 | 48 | 46 |

| | | Relative intensities | | | | | | | | |
|---|---|---|---|---|---|---|---|---|---|---|
| Relative | Mass-to-charge | 1.8 | 84.9 | 3.1 | 0.7 | 54 | 6.1 | 2.9 | 28.6 | 2 |
| intensities | ratios | 56 | 57 | 58 | 70 | 71 | 72 | 85 | 100 | 101 |
| 0.5 | 14 | 0 | 0 | 0 | 0 | 0 | 0 | 0 | 0 | 0 |
| 2.4 | 15 | 0 | 0 | 0 | 0 | 0 | 0 | 0 | 0 | 0 |
| 1.8 | 18 | 0 | 0 | 0 | 0 | 0 | 0 | 0 | 0 | 0 |
| 2.9 | 26 | 0 | 0 | 0 | 0 | 0 | 0 | 0 | 0 | 0 |
| 27.6 | 27 | 0 | 0 | 0 | 0 | 0 | 0 | 0 | 0 | 0 |
| 7.5 | 28 | 0 | 0 | 0 | 0 | 0 | 0 | 0 | 0 | 0 |
| 53 | 29 | 0 | 0 | 0 | 0 | 0 | 0 | 0 | 0 | 0 |
| 1.1 | 30 | 0 | 0 | 0 | 0 | 0 | 0 | 0 | 0 | 0 |
| 0.6 | 31 | 0 | 0 | 0 | 0 | 0 | 0 | 0 | 0 | 0 |
| 1.6 | 32 | 0 | 0 | 0 | 0 | 0 | 0 | 0 | 0 | 0 |
| 1 | 38 | 0 | 0 | 0 | 0 | 0 | 0 | 0 | 0 | 0 |
| 7.9 | 39 | 0 | 0 | 0 | 0 | 0 | 0 | 0 | 0 | 0 |
| 1.2 | 40 | 0 | 0 | 0 | 0 | 0 | 0 | 0 | 0 | 0 |
| 20.3 | 41 | 0 | 0 | 0 | 0 | 0 | 0 | 0 | 0 | 0 |
| 3.6 | 42 | 0 | 0 | 0 | 0 | 0 | 0 | 0 | 0 | 0 |
| 100 | 43 | 0 | 0 | 0 | 0 | 0 | 0 | 0 | 0 | 0 |
| 3.4 | 44 | 0 | 0 | 0 | 0 | 0 | 0 | 0 | 0 | 0 |
| 1 | 53 | 0 | 0 | 0 | 0 | 0 | 0 | 0 | 0 | 0 |
| 2.4 | 55 | 0 | 0 | 0 | 0 | 0 | 0 | 0 | 0 | 0 |
| 1.8 | 56 | 0 | 0 | 0 | 0 | 0 | 0 | 0 | 0 | 0 |
| 84.9 | 57 | 1 | 0 | 0 | 0 | 0 | 0 | 0 | 0 | 0 |
| 3.1 | 58 | 2 | 1 | 0 | 0 | 0 | 0 | 0 | 0 | 0 |
| 0.7 | 70 | 14 | 13 | 12 | 0 | 0 | 0 | 0 | 0 | 0 |
| 54 | 71 | 15 | 14 | 13 | 1 | 0 | 0 | 0 | 0 | 0 |
| 6.1 | 72 | 16 | 15 | 14 | 2 | 1 | 0 | 0 | 0 | 0 |
| 2.9 | 85 | 29 | 28 | 27 | 15 | 14 | 13 | 0 | 0 | 0 |
| 28.6 | 100 | 44 | 43 | 42 | 30 | 29 | 28 | 15 | 0 | 0 |
| 2 | 101 | 45 | 44 | 43 | 31 | 30 | 29 | 16 | 1 | 0 |

[I-3: Identification Candidate Determination of Analysis Target Substance]

The mass data of the analysis target substance prepared in I-2 (Table 2) and the mass data of the one or more reference substances prepared in I-1 (Table 1) were compared to determine an identification candidate for the analysis target substance (the analysis target substance identification candidate determining step). In this step, the threshold n=0 of the relative intensity of the ion peak was set, and the mass data of the analysis target substance consisting of the relative intensities of the threshold n or more, the mass-to-charge ratios, and the differences in the mass-to-charge ratios were compared with the mass data of the one or more reference substances prepared in I-1 to determine identification candidates for the analysis target substance. Next, the threshold n was changed to 1, 2, 3, 4, and 10 to repeatedly perform the step. With the threshold n of 0 and 1, the reference substances or the partial structures equivalent to the fragment symbols B, E, F, and M shown in Table 1 were determined as an identification candidate. With the threshold n increased to 2, since a combination (41 and 55) of the value of the mass-to-charge ratio and the value of the difference in the mass-to-charge ratios corresponding to the reference substance or the partial structure equivalent to the fragment symbol B shown in Table 1 disappeared from the mass data of the analysis target substance, the reference substances or the partial structures equivalent to the fragment symbols E, F, and M were determined as an identification candidate. With the threshold n increased to 3, only the reference substances or the partial structures equivalent to the fragment symbols E and F shown in Table 1 were determined as an identification candidate. Furthermore, with the threshold n increased to 4 and 10, since only a combination (43 and 57) of the value of the mass-to-charge ratio and the value of the difference in the mass-to-charge ratios corresponding the reference substance or the partial structure equivalent to the fragment symbol F shown in Table 1 matched the mass data of the analysis target substance, only the reference substance or the partial structure equivalent to the fragment symbol F was determined as an identification candidate. From the above-described results, the reference substance equivalent to the fragment symbol F, that is, aliphatic ketone, which was an identification candidate determined based on the mass data with larger number of ion peaks, and an identification candidate determined in case that larger value of the threshold n was set, was ranked as an identification candidate for the analysis target substance as having the increased reliability.

As a comparative example, the mass spectrum of the analysis target substance illustrated in FIG. 3 was analyzed based on the spectrum pattern (the relative intensity and m/z of each detected ion peak). Similarly to the example, the threshold n of the relative intensity of the ion peak was changed, the spectrum patterns were narrowed down to only the spectrum patterns with the ion peaks having the relative intensities of the threshold n or more, and the analysis was repeated. With the threshold n of 0, the reference substances or the partial structures equivalent to the fragment symbols B, F, M, N, and O shown in Table 1 were determined as an identification candidate. With the threshold n increased to 1 and 2, since an ion peak corresponding to the reference substance or the partial structure equivalent to the fragment symbol M shown in Table 1 disappeared from the mass spectrum of the analysis target substance, the reference substances or the partial structures equivalent to the fragment symbols B, F, N, and O were determined as an identification candidate. With the threshold n increased to 3, the reference substances or the partial structures equivalent to the fragment symbols F, N, and O shown in Table 1 were determined as an identification candidate. Furthermore, with the threshold n increased to 4 and 10, only the reference substance or the partial structure equivalent to the fragment symbol F shown in Table 1 was determined as an identification candidate. From the above-described results, the identification candidate determined by the method of the example matched the identification candidate determined by the conventional analysis method based on the spectrum pattern of the mass spectrum of the analysis target substance.

The present disclosure is not limited to the above-described example and includes various modifications. For example, the above-described example has been explained in detail for easy understanding of the description of the present disclosure, and does not necessarily include all the explained configurations. Additionally, for a part of the configurations in the respective examples, another configuration can be added, deleted, and/or replaced.

What is claimed is:

1. A method for analyzing mass spectrometry data of an analysis target substance, comprising:
acquiring mass-to-charge ratios and relative intensities of one or more detected ion peaks by measuring a mass spectrum of the analysis target substance;
preparing mass data of the analysis target substance consisting of the relative intensities, the mass-to-charge ratios, and differences in the mass-to-charge ratios of the one or more ion peaks by calculating the difference in the mass-to-charge ratios between the ion peak and another ion peak for each of the one or more detected ion peaks; and
determining an identification candidate for the analysis target substance or partial structure thereof by comparing the prepared mass data of the analysis target substance with mass data of one or more reference substances or partial structure thereof.

2. The method according to claim 1, further comprising
preparing mass data of the reference substance or partial structure thereof consisting of relative intensities, mass-to-charge ratios, and differences in the mass-to-charge ratios of one or more ion peaks for each of the one or more reference substances by measuring a mass spectrum of the one or more reference substances and calculating the difference in the mass-to-charge ratios between the one or more detected ion peaks and another ion peak for each of the one or more reference substances.

3. The method according to claim 1,
wherein the determining further comprises:
determining the identification candidates for the analysis target substance or partial structure thereof by setting a threshold n (note that n≥0) of the relative intensity of the ion peak, and comparing mass data of the analysis target substance consisting of the relative intensities of the threshold n or more, the mass-to-charge ratios, and the differences in the mass-to-charge ratios with the mass data of the one or more reference substances or partial structure thereof; and
determining a ranking of the identification candidates as having increased reliability by performing the determining of the identification candidates for the analysis target substance or partial structure thereof repeatedly while changing the threshold n within a range of 0 or more to a maximum value of the relative intensities of the one or more detected ion peaks, and selecting the identification candidate determined based on larger number of ion peaks and the identification candidate determined in case that larger value of the threshold n is set.

4. A computer program medium storing a computer program for executing the method for analyzing mass spectrometry data of an analysis target substance according to claim 1.

5. A device for analyzing mass spectrometry data of an analysis target substance, comprising:
a mass spectrum measuring unit that measures a mass spectrum of the analysis target substance;
an analysis target substance data element acquiring unit that acquires mass-to-charge ratios and relative intensities of one or more detected ion peaks in the mass spectrum measuring unit;
an analysis target substance mass data preparing unit that prepares mass data of the analysis target substance consisting of the relative intensities, the mass-to-charge ratios, and differences in the mass-to-charge ratios of the one or more ion peaks by calculating the difference in the mass-to-charge ratios between the ion peak and another ion peak for each of the one or more detected ion peaks; and
an analysis target substance identification candidate determining unit that determines an identification candidate for the analysis target substance or partial structure thereof by comparing the prepared mass data of the analysis target substance with mass data of one or more reference substances or partial structure thereof.
